(19) United States
(12) Patent Application Publication
(10) Pub. No.: US 2016/0332010 A1
(43) Pub. Date: Nov. 17, 2016

(54) Wearable Hands-Free Casualty Extraction System

(71) Applicant: Seth Alan Lowell, Macungie, PA (US)

(72) Inventor: Seth Alan Lowell, Macungie, PA (US)

(21) Appl. No.: 15/154,386

(22) Filed: May 13, 2016

Related U.S. Application Data
(60) Provisional application No. 62/161,391, filed on May 14, 2015.

(51) Int. Cl. A62B 35/00; A61B 17/132

(52) U.S. Cl. CPC A62B 35/0025; A61B 17/1322; A61B 2017/12004

(57) ABSTRACT

A wearable hands-free casualty extraction system includes a left shoulder strap, a right shoulder strap, a securing band, and a connector strap that allow the system to be worn and secured on the body. The system is hands-free and enables evacuators to perform crucial tasks such as returning fire while evacuating a wounded casualty. A first strap mount, a second strap mount, a first extraction strap, and a second extraction strap enable the system to be tethered to an evacuator during extraction. The system may include a third strap mount, a fourth strap mount, a third extraction strap, and a fourth extraction strap for further tethering capability. The system includes a first tourniquet strap and a second tourniquet strap that are worn around the legs for rapid application to a wound. The system may be tethered at the shoulders as well as at the hips.

The current application claims a priority to the U.S. Provisional Patent application Ser. No. 62/161,391 filed on May 14, 2015.

### FIELD OF THE INVENTION

The present invention relates generally to a system for extracting battlefield casualties. More specifically, the present invention is a wearable hands-free casualty extraction system for extracting wounded personnel while enabling those transporting the wounded to effectively return fire as well as perform other tasks as needed.

### BACKGROUND OF THE INVENTION

Battlefield trauma resulting in blood loss is the leading cause of death for military troops engaged in combat. While advances in technology has resulted in more effective bandages and medical procedures for wounded personnel, casualty extraction remains problematic. One means of extracting a casualty is by placing the casualty onto a litter or other suitable surface that must then be carried to the designated extraction zone by one or more troops. This is problematic for a number of reasons, chiefly the fact that time must be taken to load the casualty onto the litter, oftentimes in dangerous or exposed conditions. Additionally, the troops performing the extraction become further exposed to enemy fire while transporting the casualty and are slowed due to the burden of carrying the casualty, creating a much larger and slower target. Finally, because the casualty must be carried, the troops performing the extraction are hindered in their ability to perform tasks such as returning fire and providing cover. The inherent problems with conventional casualty extraction may often result in further casualties being taken while attempting to extract an initial casualty.

The present invention is a wearable hands-free casualty extraction system that increases the efficiency of the casualty extraction process. The hands-free nature of the present invention additionally enables troops performing an extraction to perform crucial tasks such as returning fire and providing cover rather than being completely taken out of an engagement during extraction. The present invention is designed in a manner such that major muscle groups are utilized when lifting and transporting a casualty, increasing safety and efficiency of lifting and transporting the casualty.

### BRIEF DESCRIPTION OF THE DRAWINGS

FIG. 1 is a front perspective view of the present invention.

FIG. 2 is a front perspective view of the present invention with the first tourniquet strap and the second tourniquet strap.

FIG. 3 is a front perspective view of the present invention with the first extraction strap and the second extraction strap.

FIG. 4 is a front perspective view of the present invention with the first tourniquet strap, the second tourniquet strap, the first extraction strap, the second extraction strap, the third extraction strap, and the fourth extraction strap.

FIG. 5 is a front perspective view of the present invention with the first tourniquet strap, the second tourniquet strap, and the multi-point connector strap.

FIG. 6 is a rear perspective view of the present invention with the first tourniquet strap and the second tourniquet strap.

FIG. 7 is a perspective view of the tourniquet portion of the first tourniquet strap and the second tourniquet strap.

### DETAIL DESCRIPTIONS OF THE INVENTION

All illustrations of the drawings are for the purpose of describing selected versions of the present invention and are not intended to limit the scope of the present invention.

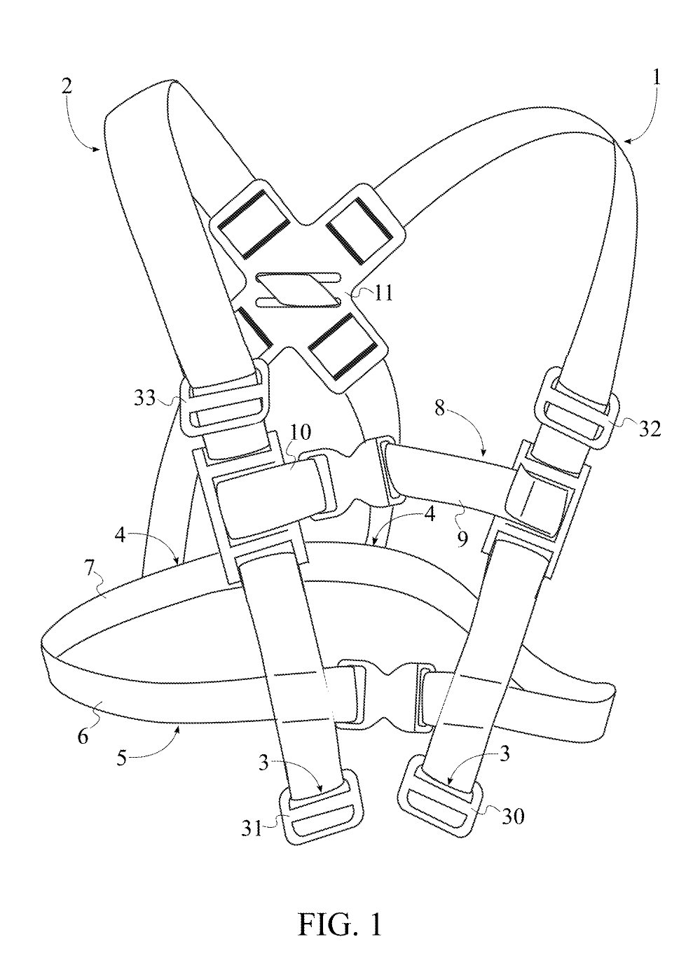

The present invention is a wearable hands-free casualty extraction system that enables tasks such as returning fire to be performed while transporting the wounded. The present invention is shown in FIG. 1 and comprises a left shoulder strap 1, a right shoulder strap 2, a securing band 5, a connector strap 8, a first strap mount 30, and a second strap mount 31. The present invention is primarily designed for use by troops while performing extraction of wounded casualties and as such is intended to be worn by all troops engaged in combat. The present invention has a wide variety of further applications as well in addition to casualty extraction.

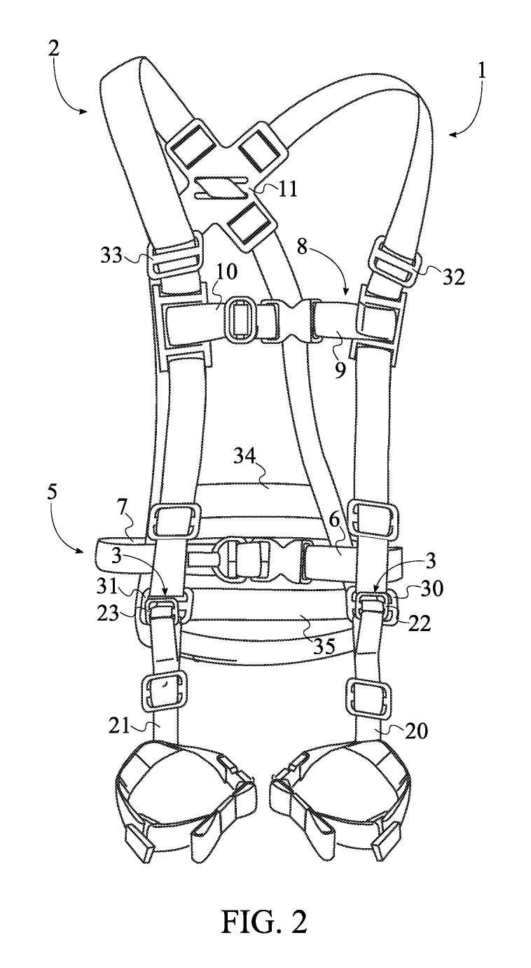

The left shoulder strap 1 and the right shoulder strap 2 enable the present invention to be worn over the user's left shoulder and right shoulder, respectively. The securing band 5 is utilized to secure the present invention in place on the user's body and is wrapped around the user's body. The securing band 5 comprises an anterior portion 6 and a posterior portion 7 corresponding respectively to the anterior region and the posterior region of the user's body. In the embodiment of the present invention shown in FIG. 1, the securing band 5 is positioned on a waist region of the user's body while in the embodiment of the present invention shown in FIG. 2, the securing band 5 is positioned on the user's abdomen. The securing band 5 may include a buckle or similar fastening mechanism for quickly detaching the present invention from the user's body. A first end 3 of the left shoulder strap 1 and a first end 3 of the right shoulder strap 2 are fixed to the anterior portion 6. Similarly, a second end 4 of the left shoulder strap 1 and a second end 4 of the right shoulder strap 2 are fixed to the posterior portion 7. The left shoulder strap 1, the right shoulder strap 2, and the securing band 5 are thus able to secure the present invention to the user's body. The first end 3 of the left shoulder strap 1 and the first end 3 of the right shoulder strap 2 are offset from each other along the anterior portion 6. Similarly, the second end 4 of the left shoulder strap 1 and the second end 4 of the right shoulder strap 2 are offset from each other along the posterior portion 7. In the preferred embodiment of the present invention, the second end 4 of the left shoulder strap 1 and the second end 4 of the right shoulder strap 2 are crossed when secured to the posterior portion 7 for further security for the present invention when worn.

The present invention further comprises a strap hub 11. In the preferred embodiment of the present invention, the strap hub 11 is utilized as a guide for the left shoulder strap 1 and the right shoulder strap 2. As previously discussed, the left shoulder strap 1 and the right shoulder strap 2 are preferably crossed. The strap hub 11 is thus preferably X-shaped. The left shoulder strap 1 and the right shoulder strap 2 traverse through the strap hub 11 in order to cross the left shoulder strap 1 and the right shoulder strap 2. The strap hub 11 is positioned adjacent to the posterior portion 7, enabling the left shoulder strap 1 and the right shoulder strap 2 to be crossed before being secured to the posterior portion 7.

The connector strap 8 is utilized for further security and stability for the present invention on the user's body. In the preferred embodiment of the present invention, the connector strap 8 functions similarly to a conventional backpack sternum strap. The connector strap 8 is connected in between the left shoulder strap 1 and the right shoulder strap 2, securing the left shoulder strap 1 and the right shoulder strap 2 together for security. Additionally, the connector strap 8 is offset from the securing band 5 along the left shoulder strap 1 and the right shoulder strap 2 in order to provide an additional securing point for the present invention on the user's body.

In the preferred embodiment of the present invention, the connector strap 8 comprises a first portion 9 and a second portion 10. The first portion 9 and the second portion 10 enable the connector strap 8 to be unfastened when detaching the present invention from the user's body. The first portion 9 is connected perpendicular to the left shoulder strap 1 while the second portion 10 is connected perpendicular to the right shoulder strap 2. This enables the left shoulder strap 1 and the right shoulder strap 2 to be joined via the first portion 9 and the second portion 10. The first portion 9 is removably engaged to the second portion 10, enabling the connector strap 8 to be quickly unfastened when removing the present invention from the user's body. The first portion 9 and the second portion 10 thus enable the connector strap 8 to be utilized in a similar manner as a conventional backpack sternum strap.

The present invention further comprises a first strap mount 30 and a second strap mount 31. The first strap mount 30 and the second strap mount 31 are utilized when tethering one instance of the present invention to another instance of the present invention during extraction. The first strap mount 30 is connected onto the first end 3 of the left shoulder strap 1, adjacent to the securing band 5. Similarly, the second strap mount 31 is connected onto the first end 3 of the right shoulder strap 2, adjacent to the securing band 5. The first strap mount 30 and the second strap mount 31 are thus positioned adjacent to the user's waist or abdomen. In the preferred embodiment of the present invention, the first strap mount 30 and the second strap mount 31 are D-rings or similar fasteners.

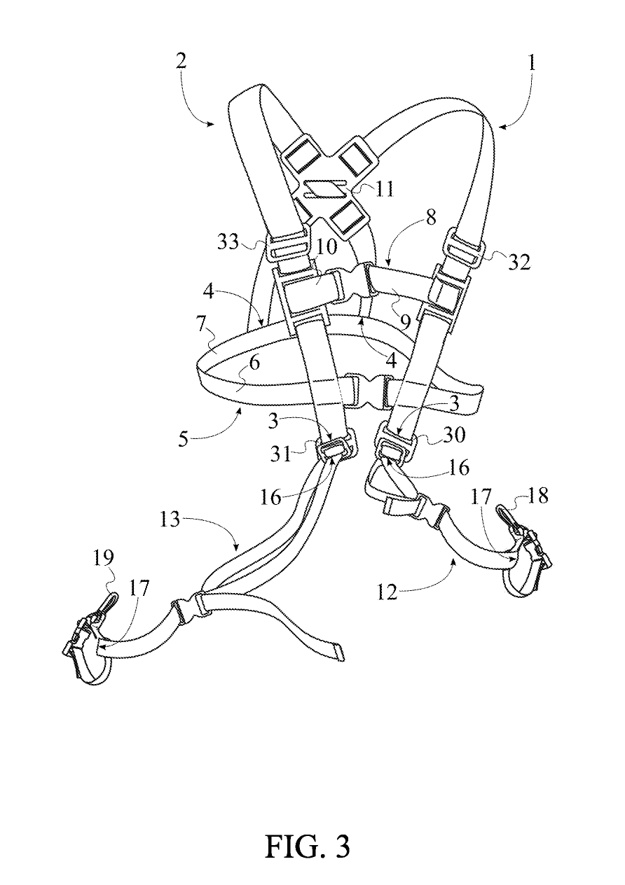

As shown in FIG. 3, the present invention may be utilized in conjunction with a first extraction strap 12 and a second extraction strap 13. The first extraction strap 12 and the second extraction strap 13 are utilized to tether one instance of the present invention to another instance of the present invention. Alternatively, the first extraction strap 12 and the second extraction strap 13 may be tethered to litters, body armor drag handles, belts, and similar accessories. During use of the present invention, a proximal end 16 of the first extraction strap 12 is removably connected to the first strap mount 30 while a proximal end 16 of the second extraction strap 13 is removably connected to the second strap mount 31. This enables the user to tether to a wounded casualty as well as quickly disconnect from the wounded casualty if necessary. The present invention further comprises a first quick-release fastener 18 and a second quick-release fastener 19. In the embodiment of the present invention shown in FIG. 3, the first quick-release fastener 18 and the second quick-release fastener 19 are utilized to separate the first extraction strap 12 and the second extraction strap 13 into two separate halves. The first quick-release fastener 18 is coupled adjacent to a distal end 17 of the first extraction strap 12 while the second quick-release fastener 19 is coupled adjacent to a distal end 17 of the second extraction strap 13. The distal end 17 of the first extraction strap 12 and the distal end 17 of the second extraction strap 13 may thus be separated from the proximal end 16 of the first extraction strap 12 and the proximal end 16 of the second extraction strap 13, respectively. The lengths of the first extraction strap 12 and the second extraction strap 13 are adjustable.

The present invention further comprises a first hip connector 22 and a second hip connector 23. The first hip connector 22 and the second hip connector 23 enable the first extraction strap 12 and the second extraction strap 13 to be joined to the left shoulder strap 1 and the right shoulder strap 2. The first hip connector 22 adjacently couples the proximal end 16 of the first extraction strap 12 to the first strap mount 30. Similarly, the second hip connector 23 adjacently couples the proximal end 16 of the second extraction strap 13 to the second strap mount 31. The first hip connector 22 and the second hip connector 23 are thus able to interface with the first strap mount 30 and the second strap mount 31, respectively. This additionally enables the first extraction strap 12 and the second extraction strap 13 to easily be separated from the left shoulder strap 1 and the right shoulder strap 2 as needed. In the preferred embodiment of the present invention, the first hip connector 22 and the second hip connector 23 are carabiners or similar fasteners for interfacing with the first strap mount 30 and the second strap mount 31.

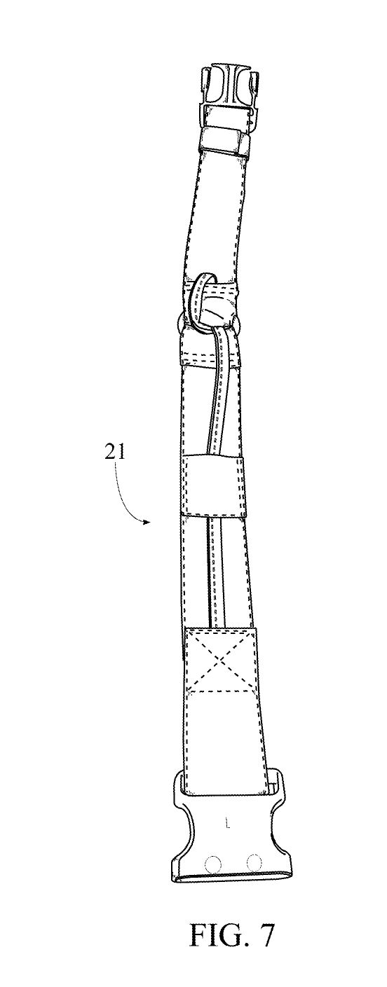

The embodiment of the present invention shown in FIG. 2 and FIG. 7 further comprises a first tourniquet strap 20 and a second tourniquet strap 21 that are utilized to apply pressure to a wound in order to stem blood flow. The first tourniquet strap 20 and the second tourniquet strap 21 are designed to be worn on the user's legs when the present invention is in use. In this embodiment of the present invention, the first tourniquet strap 20 is adjacently connected to the first strap mount 30 while the second tourniquet strap 21 is adjacently connected to the second strap mount 31. The first tourniquet strap 20 and the second tourniquet strap 21 may thus be secured to the left shoulder strap 1 and the right shoulder strap 2, allowing the present invention to be worn as a harness.

As shown in FIG. 3, the first extraction strap 12 and the second extraction strap 13 may be utilized during extraction of a casualty. The proximal end 16 of the first extraction strap 12 is adjacently connected to the first tourniquet strap 20 while the proximal end 16 of the second extraction strap 13 is adjacently connected to the second tourniquet strap 21. The first tourniquet strap 20 and the second tourniquet strap 21 may thus be utilized to tether to the first extraction strap 12 and the second extraction strap 13. A fastener or similar tethering device may be present on the first tourniquet strap 20 and the second tourniquet strap 21.

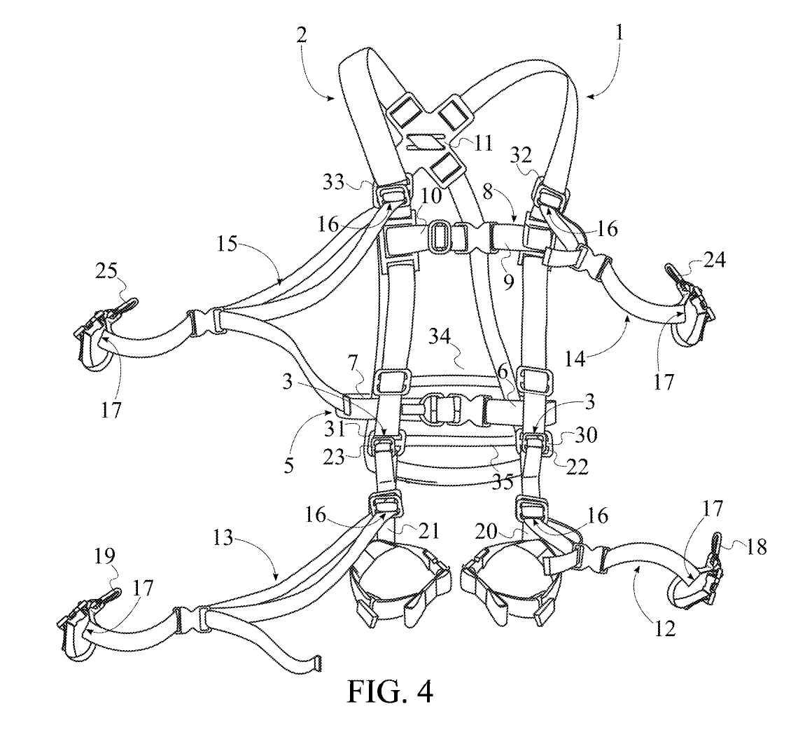

The embodiment of the present invention shown in FIG. 4 further comprises a third extraction strap 14, a fourth extraction strap 15, a third strap mount 32, and a fourth strap mount 33. The third extraction strap 14 and the fourth extraction strap 15 function in the same manner as the first extraction strap 12 and the second extraction strap 13. The third strap mount 32 and the fourth strap mount 33 function in the same manner as the first strap mount 30 and the second strap mount 31 and provide mounting points for the third extraction strap 14 and the fourth extraction strap 15. The third extraction strap 14, the fourth extraction strap 15, the third strap mount 32, and the fourth strap mount 33 are primarily intended for use when two users of the present invention are transporting a wounded casualty. The third extraction strap 14 is connected onto the left shoulder strap 1, in between the connector strap 8 and the second end 4 of the left shoulder strap 1. Similarly, the fourth extraction strap 15 is connected onto the right shoulder strap 2, in between the connector strap 8 and the second end 4 of the right shoulder strap 2. The lengths of the third extraction strap 14 and the fourth extraction strap 15 are adjustable as well. The third strap mount 32 and the fourth strap mount 33 enable the third extraction strap 14 and the fourth extraction strap 15 to be tethered to the left shoulder strap 1 and the right shoulder strap 2 on the user's shoulders. In conjunction with the first strap mount 30 and the second strap mount 31, the third strap mount 32 and the fourth strap mount 33 enable one instance of the present invention to tether to another two instances of the present invention. The third strap mount 32 is positioned adjacent to the connector strap 8 while the fourth strap mount 33 is positioned adjacent to the connector strap 8 as well. This ensures that the third strap mount 32 and the fourth strap mount 33 are positioned towards the anterior region of the user's body, similar to the first strap mount 30 and the second strap mount 31.

The present invention further comprises a third quick-release fastener 24 and a fourth quick-release fastener 25. The third quick-release fastener 24 and the fourth quick-release fastener 25 function in the same manner as the first quick-release fastener 18 and the second quick-release fastener 19. As such, the third extraction strap 14 and the fourth extraction strap 15 may each be separated into two separate halves. A proximal end 16 of the third extraction strap 14 is removably connected to the third strap mount 32 while a proximal end 16 of the fourth extraction strap 15 is removably connected to the fourth strap mount 33. The third strap mount 32 and the fourth strap mount 33 may thus be utilized to tether a wounded casualty during evacuation. The third strap mount 32 and the fourth strap mount 33 enable tethering of a wounded casualty's shoulders in addition to tethering of the hips via the first strap mount 30 and the second strap mount 31. The third quick-release fastener 24 is coupled adjacent to a distal end 17 of the third extraction strap 14 while the fourth quick-release fastener 25 is coupled adjacent to a distal end 17 of the fourth extraction strap 15. The distal end 17 of the third extraction strap 14 and the distal end 17 of the fourth extraction strap 15 may thus be separated from the proximal end 16 of the third extraction strap 14 and the proximal end 16 of the fourth extraction strap 15, respectively.

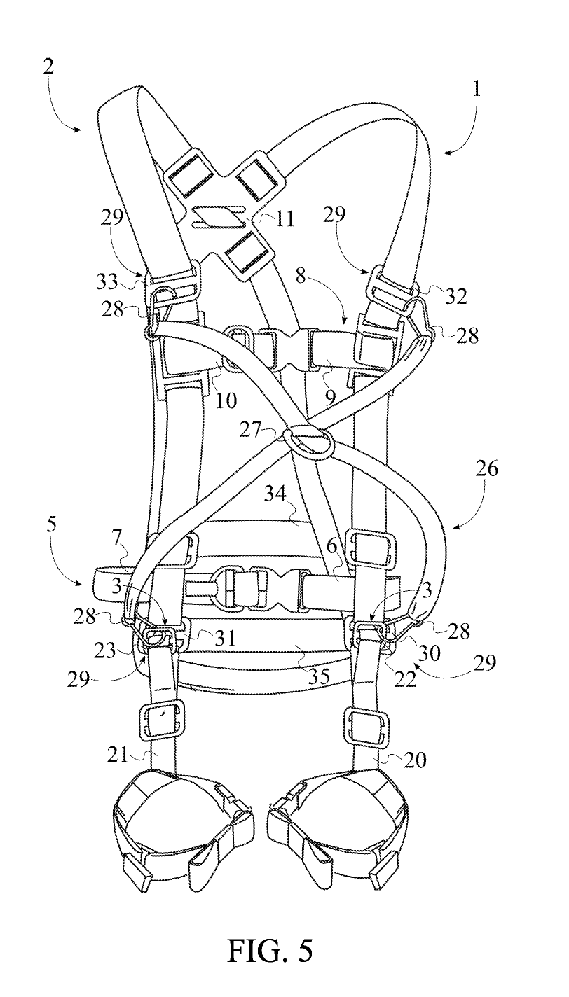

In addition to the first extraction strap 12, the second extraction strap 13, the third extraction strap 14, and the fourth extraction strap 15, a multi-point connector strap 26 may be utilized to tether to the present invention as shown in FIG. 5. In the preferred embodiment of the present invention, the multi-point connector strap 26 is utilized to tether the present invention to a helicopter extraction line or a similar connection point. The multi-point connector strap 26 comprises a primary fastener 27 that is attachable to the helicopter extraction line or similar mounting point. The multi-point connector strap 26 further comprises a plurality of secondary fasteners 28 that is designed to interface with a plurality of strap mounts 29. The plurality of strap mounts 29 comprises the first strap mount 30, the second strap mount 31, the third strap mount 32, and the fourth strap mount 33. As such, the multi-point connector strap 26 may be connected to the user's shoulders and the user's hips. The plurality of secondary fasteners 28 is radially tethered to the primary fastener 27, positioning the primary fastener 27 in between the plurality of secondary fasteners 28 and enabling a wounded casualty to be safely and securely lifted when attached to a helicopter extraction line. Each of the plurality of secondary fasteners 28 is coupled adjacent to a corresponding mount from the plurality of strap mounts 29. This enables the multi-point connector strap 26 to be secured to a wounded casualty's hips and shoulders during extraction and eliminates the need for a backboard or similar structure for the wounded casualty.

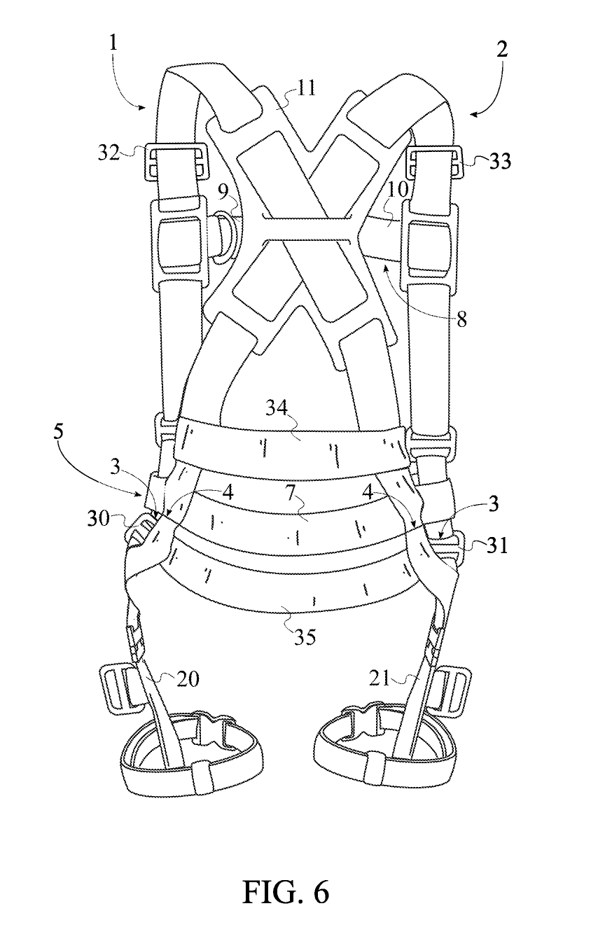

As shown in FIG. 6, the present invention further comprises a first back support strap 34 and a second back support strap 35. The first back support strap 34 and the second back support strap 35 provide lower back support by bracing the lower back muscles when the present invention is worn. This is crucial to those who are transporting a wounded casualty as the first back support strap 34 and the second back support strap 35 are able to reduce the likelihood of lower back injury. The first back support strap 34 and the second back support strap 35 are connected in between the left shoulder strap 1 and the right shoulder strap 2, adjacent to the securing band 5. The first back support strap 34 and the second back support strap 35 are thus secured to both the left shoulder strap 1 and the right shoulder strap 2, allowing the first back support strap 34 and the second back support strap 35 to serve as braces on the user's lower back muscles. The first back support strap 34 and the second back support strap 35 are positioned adjacent to the securing band 5, allowing the securing band 5 to provide lower back support in conjunction with the first back support strap 34 and the second back support strap 35. The posterior portion 7 is positioned in between the first back support strap 34 and the second back support strap 35, positioning the first back support strap 34 and the second back support strap 35 adjacent to the lower back muscles when the present invention is worn. The first back support strap 34, the second back support strap 35, and the posterior portion 7 are oriented parallel to each other. The first back support strap 34, the second back support strap 35, and the posterior portion 7 are thus able to function in a manner similar to a lumbar support belt.

The present invention is primarily intended for use in transporting wounded military personnel while leaving the hands of those transporting the wounded free to perform tasks such as returning fire. Accuracy and precision when discharging a firearm is a safety priority for the shooter as well as to avoid collateral damage. The present invention may be utilized in a variety of casualty extraction scenarios. For example, the present invention may be utilized by one user or multiple users in order to extract a wounded casualty. When multiple users are transporting a wounded casualty, it is important that the casualty remains level. Because a casualty that is geared with a full combat load may weigh over 300 pounds, the present invention is designed in a manner such that bone structure and major muscles groups are activated and utilized when lifting a wounded casualty. The present invention enables a wounded casualty to be quickly tethered and moved out of the line of fire. The present invention has additional applications as well. One example application is K9 operations in which the present invention is tethered to a K9 unit, freeing the user's hands to accurately fire a weapon. Similarly, the present invention may be utilized in prisoner handling applications, leaving the user's hands free to perform other tasks while detaining a prisoner. The present invention additionally may be utilized for non-lifesaving applications. For example, the present invention may be utilized in loadbearing applications for transporting heavy equipment. The present invention additionally enables rappelling via the plurality of strap mounts 29 that may be utilized to tether the present invention to a rappelling rope. The first strap mount 30, the second strap mount 31, the third strap mount 32, and the fourth strap mount 33 enable rappelling gear to be tethered to the present invention on the user's shoulders and/or the user's hips. The first extraction strap 12, the second extraction strap 13, the third extraction strap 14, and the fourth extraction strap 15 are available in multiple lengths and may be utilized as rifle slings.

Although the present invention has been explained in relation to its preferred embodiment, it is understood that many other possible modifications and variations can be made without departing from the spirit and scope of the present invention as hereinafter claimed.

What is claimed is:

1. A wearable hands-free casualty extraction system comprises:
a left shoulder strap;
a right shoulder strap;
a securing band;
a connector strap;
a first strap mount;
a second strap mount;
the securing band comprises an anterior portion and a posterior portion;
a first end of the left shoulder strap and a first end of the right shoulder strap being fixed to the anterior portion;
a second end of the left shoulder strap and a second end of the right shoulder strap being fixed to the posterior portion;
the first end of the left shoulder strap and the first end of the right shoulder strap being offset from each other along the anterior portion;
the second end of the left shoulder strap and the second end of the right shoulder strap being offset from each other along the posterior portion;
the connector strap being connected in between the left shoulder strap and the right shoulder strap;
the connector strap being offset from the securing band along the left shoulder strap and the right shoulder strap;
the first strap mount being connected onto the first end of the left shoulder strap, adjacent to the securing band; and
the second strap mount being connected onto the first end of the right shoulder strap, adjacent to the securing band.

2. The wearable hands-free casualty extraction system as claimed in claim 1 further comprises:
a strap hub;
the left shoulder strap and the right shoulder strap traversing through the strap hub; and
the strap hub being positioned adjacent to the posterior portion.

3. The wearable hands-free casualty extraction system as claimed in claim 1 further comprises:
the connector strap comprises a first portion and a second portion;
the first portion being connected perpendicular to the left shoulder strap;
the second portion being connected perpendicular to the right shoulder strap; and
the first portion being removably engaged to the second portion.

4. The wearable hands-free casualty extraction system as claimed in claim 1 further comprises:
a first extraction strap;
a second extraction strap;
a first quick-release fastener;
a second quick-release fastener;
a proximal end of the first extraction strap being removably connected to the first strap mount;
a proximal end of the second extraction strap being removably connected to the second strap mount;
the first quick-release fastener being coupled adjacent to a distal end of the first extraction strap; and
the second quick-release fastener being coupled adjacent to a distal end of the second extraction strap.

5. The wearable hands-free casualty extraction system as claimed in claim 4 further comprises:
a first hip connector;
a second hip connector;
the first hip connector adjacently coupling the proximal end of the first extraction strap to the first strap mount; and
the second hip connector adjacently coupling the proximal end of the second extraction strap to the second strap mount.

6. The wearable hands-free casualty extraction system as claimed in claim 1 further comprises:
a first tourniquet strap;
a second tourniquet strap;
the first tourniquet strap being adjacently connected to the first strap mount; and
the second tourniquet strap being adjacently connected to the second strap mount.

7. The wearable hands-free casualty extraction system as claimed in claim 6 further comprises:
a first extraction strap;
a second extraction strap;
a first quick-release fastener;
a second quick-release fastener;
a proximal end of the first extraction strap being adjacently connected to the first tourniquet strap;
a proximal end of the second extraction strap being adjacently connected to the second tourniquet strap;
the first quick-release fastener being coupled adjacent to a distal end of the first extraction strap; and
the second quick-release fastener being coupled adjacent to a distal end of the second extraction strap.

8. The wearable hands-free casualty extraction system as claimed in claim 1 further comprises:
a third extraction strap;
a fourth extraction strap;
a third strap mount;
a fourth strap mount;
the third strap mount being connected onto the left shoulder strap, in between the connector strap and the second end of the left shoulder strap;
the fourth strap mount being connected onto the right shoulder strap, in between the connector strap and the second end of the right shoulder strap;
the third strap mount being positioned adjacent to the connector strap; and
the fourth strap mount being positioned adjacent to the connector strap.

9. The wearable hands-free casualty extraction system as claimed in claim 8 further comprises:
a third quick-release fastener;
a fourth quick-release fastener;
a proximal end of the third extraction strap being removably connected to the third strap mount;
a proximal end of the fourth extraction strap being removably connected to the fourth strap mount;
the third quick-release fastener being coupled adjacent to a distal end of the third extraction strap; and
the fourth quick-release fastener being coupled adjacent to a distal end of the fourth extraction strap.

10. The wearable hands-free casualty extraction system as claimed in claim 8 further comprises:
a multi-point connector strap;
a plurality of strap mounts;
the multi-point connector strap comprises a primary fastener and a plurality of secondary fasteners;
the plurality of strap mounts comprises the first strap mount, the second strap mount, the third strap mount, and the fourth strap mount;
the plurality of secondary fasteners being radially tethered to the central fastener; and
each of the plurality of secondary fasteners being coupled adjacent to a corresponding mount from the plurality of strap mounts.

11. The wearable hands-free casualty extraction system as claimed in claim 1 further comprises:
a first back support strap;
a second back support strap;
the first back support strap and the second back support strap being connected in between the left shoulder strap and the right shoulder strap, adjacent to the securing band;
the posterior portion being positioned in between the first back support strap and the second back support strap; and
the first back support strap, the second back support strap, and the posterior portion being oriented parallel to each other.
